(19) United States
(12) Patent Application Publication
(10) Pub. No.: US 2007/0191459 A1
(43) Pub. Date: Aug. 16, 2007

(54) Use of Benzo-Heteroaryl Sulfamide Derivatives for Lowering Lipids and Lowering Blood Glucose Levels

(76) Inventor: Virginia L. Smith-Swintosky, Hatfield, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/673,709

(22) Filed: Feb. 12, 2007

Related U.S. Application Data
(60) Provisional application No. 60/773,808, filed on Feb. 15, 2006.

(51) Int. Cl. A61K 31/38

(52) U.S. Cl. 514/415; 514/443; 514/469; 514/709

(57) ABSTRACT

The present invention is a method for the glucose related disorders and lipid related disorders comprising administering to a subject in need thereof a therapeutically effective amount of one or more novel b benzo-heteroaryl sulfamide derivatives of formula (I) as herein defined. The present invention is further directed to methods of treatment comprising co-therapy with an anti-diabetic agent, and anti-lipid agent and/or an anti-obesity agent.

### CROSS REFERENCE TO RELATED APPLICATIONS

The application claims the benefit of U.S. Provisional Application 60/773,808, filed on Feb. 15, 2006, which is incorporated by reference herein in it's entirety.

### FIELD OF THE INVENTION

The present invention is directed to the use of benzo-heteroaryl sulfamide derivatives for lowering lipids, lowering blood glucose levels, improving glycemic control, treating Type II diabetes mellitis, metabolic syndrome, hyperglycemia and related disorders.

### BACKGROUND OF THE INVENTION

Diabetes mellitus is a medical term for the presence of elevated blood glucose. People with diabetes either don't produce insulin, produce too little insulin or do not respond to insulin, resulting in the build up of glucose in the blood. The most common form of diabetes is Type 2 diabetes, once referred to as adult onset diabetes or non-insulin dependent diabetes (NIDDM), which may account for >90% of diabetes in adults. However, as the younger population becomes increasingly overweight or obese, Type 2 diabetes is becoming more prevalent in teens and children. Diabetes may also refer to gestational diabetes, Type 1 diabetes or autoimmune diabetes, once referred to as juvenile onset diabetes and type 1½ diabetes, also referred to as latent-autoimmune diabetes in adults or LADA. Diabetes may occur because of poor dietary habits or lack of physical activity (e.g., sedentary lifestyle), genetic mutations, injury to the pancreas, drug (e.g., AIDS therapies) or chemical (e.g., steroid) exposure or disease (e.g., cystic fibrosis, Down syndrome, Cushing's syndrome). Two rare types of genetic defects leading to diabetes are termed maturity-onset diabetes of the young (MODY) and atypical diabetes mellitus (ADM).

Type II diabetes mellitus (non-insulin-dependent diabetes mellitus or NIDDM) is a metabolic disorder involving disregulation of glucose metabolism and insulin resistance, and long-term complications involving the eyes, kidneys, nerves, and blood vessels. Type II diabetes mellitus usually develops in adulthood (middle life or later) and is described as the body's inability to make either sufficient insulin (abnormal insulin secretion) or its inability to effectively use insulin (resistance to insulin action in target organs and tissues). More particularly, patients suffering from Type II diabetes mellitus have a relative insulin deficiency. That is, in these patients, plasma insulin levels are normal to high in absolute terms, although they are lower than predicted for the level of plasma glucose that is present.

Type II diabetes mellitus is characterized by the following clinical signs or symptoms: persistently elevated plasma glucose concentration or hyperglycemia; polyuria; polydipsia and/or polyphagia; chronic microvascular complications such as retinopathy, nephropathy and neuropathy; and macrovascular complications such as hyperlipidemia and hypertension which can lead to blindness, end-stage renal disease, limb amputation and myocardial infarction.

Syndrome X, also termed Insulin Resistance Syndrome (IRS), Metabolic Syndrome, or Metabolic Syndrome X, is a disorder that presents risk factors for the development of Type II diabetes mellitus and cardiovascular disease including glucose intolerance, hyperinsulinemia and insulin resistance, hypertriglyceridemia, hypertension and obesity.

The diagnosis of Type II diabetes mellitus includes assessment of symptoms and measurement of glucose in the urine and blood. Blood glucose level determination is necessary for an accurate diagnosis. More specifically, fasting blood glucose level determination is a standard approach used. However, the oral glucose tolerance test (OGTT) is considered to be more sensitive than fasted blood glucose level. Type II diabetes mellitus is associated with impaired oral glucose tolerance (OGT). The OGTT thus can aid in the diagnosis of Type II diabetes mellitus, although generally not necessary for the diagnosis of diabetes (Emancipator K, Am J Clin Pathol 1999 November; 112(5):665-74; Type 2 Diabetes Mellitus, Decision Resources Inc., March 2000). The OGTT allows for an estimation of pancreatic beta-cell secretory function and insulin sensitivity, which helps in the diagnosis of Type II diabetes mellitus and evaluation of the severity or progression of the disease (e.g., Caumo A, Bergman R N, Cobelli C,. J Clin Endocrinol Metab 2000, 85(11):4396-402). More particularly, the OGTT is extremely helpful in establishing the degree of hyperglycemia in patients with multiple borderline fasting blood glucose levels that have not been diagnosed as diabetics. In addition, the OGTT is useful in testing patients with symptoms of Type II diabetes mellitus where the possible diagnosis of abnormal carbohydrate metabolism has to be clearly established or refuted.

Thus, impaired glucose tolerance is diagnosed in individuals that have fasting blood glucose levels less than those required for a diagnosis of Type II diabetes mellitus, but have a plasma glucose response during the OGTT between normal and diabetics. Impaired glucose tolerance is considered a prediabetic condition, and impaired glucose tolerance (as defined by the OGTT) is a strong predictor for the development of Type II diabetes mellitus (Haffner S M, Diabet Med 1997 August; 14 Suppl 3:S12-8).

Type II diabetes mellitus is a progressive disease associated with the reduction of pancreatic function and/or other insulin-related processes, aggravated by increased plasma glucose levels. Thus, Type II diabetes mellitus usually has a prolonged prediabetic phase and various pathophysiological mechanisms can lead to pathological hyperglycemia and impaired glucose tolerance, for instance, abnormalities in glucose utilization and effectiveness, insulin action and/or insulin production in the prediabetic state (Goldberg R B, Med Clin North Am 1998 July; 82(4):805-21).

The prediabetic state associated with glucose intolerance can also be associated with a predisposition to abdominal obesity, insulin resistance, hyperlipidemia, and high blood pressure, that is, Syndrome X (Groop L, Forsblom C, Lehtovirta M, Am J Hypertens 1997 September; 10(9 Pt 2):172S180S; Haffner S M, J Diabetes Complications 1997 March-April; 11(2):69-76; Beck-Nielsen H, Henriksen J E, Alford F, Hother-Nielson O, Diabet Med 1996 September; 13(9 Suppl 6):S78-84).

Thus, defective carbohydrate metabolism is pivotal to the pathogenesis of Type II diabetes mellitus and impaired glucose tolerance (Dinneen S F, Diabet Med 1997 August; 14 Suppl 3:S19-24). In fact, a continuum from impaired glucose tolerance and impaired fasting glucose to definitive Type II diabetes mellitus exists (Ramlo-Halsted B A, Edelman S V, Prim Care 1999 December; 26(4):771-89).

Early intervention in individuals at risk to develop Type II diabetes mellitus, focusing on reducing the pathological hyperglycemia or impaired glucose tolerance may prevent or delay the progression towards Type II diabetes mellitus and associated complications and/or Syndrome X. Therefore, by effectively treating impaired oral glucose tolerance and/or elevated blood glucose levels, one can prevent or inhibit the progression of the disorder to Type II diabetes mellitus or Syndrome X.

Dyslipidemia is a group of diseases characterized by abnormal changes or levels in concentrations of lipoproteins and associated lipids, such as triglyceride and cholesterol, in the blood. Lipids are transported through the bloodstream in the form of lipoproteins consisting essentially of a core of apolar molecules such as triglyceride and cholesterol ester surrounded by an envelope of amphipathic lipids, primarily phospholipids. Acquired hyperlipidemia/hyperlipoproteinemia develops as a consequence of dietary imbalance, drug or compound effects, or disease, such as thyroid deficiency or diabetes. Familial hyperlipidemia/hyperlipoproteinemia is characterized by autosomal inheritance and is associated with an increase in lipoprotein and lipid content in the blood. Familial hyperlipidemia/hyperlipoproteinemia is subdivided into to five categories (types I-V) depending on the composition and type of lipoprotein particles in the blood. For example, in Type I and Type IV hyperlipoproteinemia, triglyceride is elevated predominately in chylomicron and VLDL particles, respectively. In general, there is an inverse relation between HDL-cholesterol and triglyceride levels that contributes to dyslipidemia. If left untreated, dyslipidemia (e.g., low HDL-cholesterol and high triglyceride or LDL-cholesterol levels) can exacerbate other conditions, such as pancreatitis, abnormal glucose tolerance, diabetes, coronary artery disease, ischemic heart diseases, atherosclerosis, hepatosplenomegaly, and fatty liver disease.

There remains a need to provide an effective treatment for glucose related disorders such as elevated glucose levels, Type II diabetes mellitus, Syndrome X, and the like. There also remains need to provide an effective treatment for lipid related disorders such as elevated glucose levels, dyslipidemia, and the like.

### SUMMARY OF THE INVENTION

The present invention is directed to a method for a method for the treatment of glucose related disorders and/or lipid related disorders comprising administering to a subject in need thereof a therapeutically effective amount of a compound of formula (I)

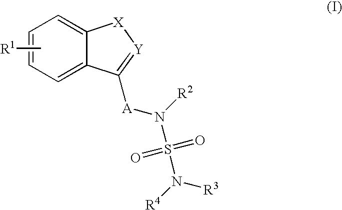

wherein

R¹ is selected from the group consisting of hydrogen, halogen, hydroxy, methoxy, trifluoromethyl, nitro and cyano;

X-Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is selected from the group consisting of hydrogen and methyl;

R³ and R⁴ are each independently selected from the group consisting of hydrogen and C₁₋₄alkyl;

alternatively, R³ and R⁴ are taken together with the nitrogen atom to which they are bound to form a 5 to 7 membered, saturated, partially unsaturated or aromatic ring structure, optionally containing one to three additional heteroatoms independently selected from the group consisting of O, N and S;

or a pharmaceutically acceptable salt thereof.

Exemplifying the invention is a method of treating glucose related disorders comprising administering to a subject in need thereof a therapeutically effective amount of any of the compounds described above. In another example, the invention is directed to a method of treating lipid related disorders comprising administering to a subject in need thereof a therapeutically effective amount of any of the compounds described above.

The present invention is further directed to a method for the treatment of a glucose related disorder comprising co-therapy with at least one anti-diabetic agent and a compound of formula (I) as described herein. The present invention is further directed to a method for the treatment of a lipid related disorder comprising co-therapy with at least one anti-lipid agent and a compound of formula (I) as described herein. The present invention is further directed to a method for the treatment of a glucose related disorder or a lipid-related disorder comprising co-therapy with at least one anti-diabetic agent and/or at least one anti-lipid agent and a compound of formula (I) as described herein. The present invention is further directed to a method for the treatment of a glucose-related disorder comprising co-therapy with an anti-obesity agent and a compound of formula (I)) as described herein.

### DETAILED DESCRIPTION OF THE INVENTION

The present invention is directed to a method for the treatment of glucose related disorders and/or lipid related disorders comprising administering to a subject in need thereof a therapeutically effective amount of a compound of formula (I)

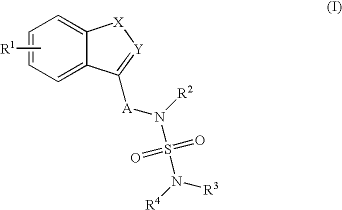

or a pharmaceutically acceptable salt thereof, wherein R¹, R², R³, R⁴, —X—Y— and A are as herein defined.

The present invention is further directed to methods for the treatment of glucose related disorders and lipid related disorders comprising co-therapy with at least one anti-diabetic and/or at least one anti-lipid agent and a compound of formula (I) as described herein.

One skilled in the art will recognize that treatment of glucose related disorders and/or lipid-related disorders may further benefit from treatment of co-morbid overweight and obesity conditions. Thus, in an embodiment, the methods of the present invention comprise co-therapy with an anti-obesity agent and a compound of formula (I) as described herein.

As used herein, the term “glucose related disorder” shall be defined as any disorder which is characterized by elevated glucose levels. Glucose related disorders include elevated glucose level, pre-diabetes, impaired oral glucose tolerance, poor glycemic control, Type II Diabetes Mellitus, Syndrome X (also known as metabolic syndrome), gestational diabetes, insulin resistance, hyperglycemia and loss of muscle mass as a results of hyperglycemia (cachexia).

Treatment of glucose related disorders may comprise lowering glucose levels, improving glycemic control, decreasing insulin resistance and/or preventing the development of a glucose related disorder (for example preventing a patient suffering from impaired oral glucose tolerance or elevated glucose levels from developing Type II diabetes mellitus).

As used herein, the term “lipid related disorder” shall be defined as any disorder which is characterized by non-normal lipid levels. Lipid related disorders include elevated triglyceride levels, low HDL cholesterol and dyslipidemia, preferably elevated triglyceride levels or low HDL cholesterol levels Treatment of lipid related disorder may comprise lowering triglycerides, elevating HDL cholesterol and/or improving the triglyceride/HDL ratio.

As used herein, the term “anti-diabetic agent” shall mean any pharmaceutical agent which decreases blood levels, improves glycemic control and/or improves insulin sensitivity. Anti-diabetic agents useful for the treatment of Type II diabetes mellitus and Syndrome X include, but are not limited to, sulfonylureas, meglitinides, agents which modify insulin secretion, biguanides, thiazolidinediones, PPAR-gamma agonists, Retinoid-X receptor (RXR) modulators, insulin sensitizing agents, alpha-glucosidase inhibitors, insulins, small molecule mimics of insulin, Na-glucose co-transporter inhibitors, amylin agonists, glucagon antagonists, GLP-1 and GLP-1 analogs, DPPIV inhibitors, and the like.

Suitable examples of anti-diabetic agents include, exenatide, chlorpropamide, tolazamide, tolbutamide, glyburide, glipizide, glimepiride, repaglinide, metformin, rosiglitazone, pioglitazone, troglitazone, isaglitazone (known as MCC-555), 2-[2-[(2R)-4-hexyl-3,4-dihydro-3-oxo-2H-1,4-benzoxazin-2-yl]ethoxy]-benzene acetic acid, GW2570, targretin, 9-cis-retinoic acid, ascarbose, miglitol, L-783281, TE-17411, T-1095, BAY-279955, phlorizen, pramlintide, regular-acting insulin, short-acting insulin, intermediate-acting insulin, long-acting insulin, inhaled insulin, insulin analogues, acetohexamide, buformin, glibomuride, glyhexamide, glymidine, linogliride, palmoxirate, zopolrestat; etoformin, gllicalzide, glypinamide, and the like.

More particularly, anti-diabetic agents include, but are not limited to:

(a) Sulfonylureas, which increase insulin production by stimulating pancreatic beta cells, and therefore act as insulin secretagogues. The primary mechanism of action of sulfonylureas is to close ATP-sensitive potassium channels in the beta-cell plasma membrane, initiating a chain of events that result in insulin release. Suitable examples of sulfonylureas include, but are not limited to chlorpropamide, tolazamide, tolbutamide, glyburide, glipizide, glimepiride, and like;

(b) Meglitinides, another class of insulin secretagogues, that have a mechanism of action distinct from that of the sulfonylureas. Suitable examples of meglitinides include, but are not limited to repaglinide;

(c) Agents which modify insulin secretion such as Glucagon-like Peptide-1(GLP-1) and it's mimetics, Glucose-insulinotropic peptide (GIP) and it's mimetics, Exendin and it's mimetics, and Dipeptyl Protease Inhibitors (DPPIV);

(d) Biguanides which decrease liver glucose production and increase the uptake of glucose. Suitable examples include, but are not limited to metformin;

(e) Thiazolidinediones, insulin sensitizing drugs which decrease peripheral insulin resistance by enhancing the effects of insulin at target organs and tissues. These drugs bind and activate the nuclear receptor, peroxisome proliferator-activated receptor-gamma (PPAR-gamma) which increases transcription of specific insulin-responsive genes. Suitable examples of PPAR-gamma agonists are the thiazolidinediones which include, but are not limited to rosiglitazone, pioglitazone, troglitazone, isaglitazone (known as MCC-555), 2-[2-[(2R)-4-hexyl-3,4-dihydro-3-oxo-2H-1,4-benzoxazin-2-yl]ethoxy]-benzene acetic acid, and the like. Additionally, the non-thiazolidinediones also act as insulin sensitizing drugs, and include, but are not limited to GW2570, and the like;

(f) Retinoid-X receptor (RXR) modulators, also insulin sensitizing drugs, which include, but are not limited to targretin, 9-cis-retinoic acid, and the like;

(g) Other insulin sensitizing agents include, but are not limited to INS-1, PTP-1B inhibitors, GSK3 inhibitors, glycogen phosphorylase a inhibitors, fructose-1,6-bisphosphatase inhibitors, and the like;

(h) Alpha-glucosidase inhibitors which act to inhibit alpha-glucosidase. Alpha-glucosidase converts fructose to glucose, thus these inhibitors delay the digestion of carbohydrates. The undigested carbohydrates are subsequently broken down in the gut, thereby reducing the post-prandial glucose peak. Suitable examples include, but are not limited to, acarbose and miglitol;

(i) Insulins, including regular or short-acting, intermediate-acting, and long-acting insulins, inhaled insulin and insulin analogues such as insulin molecules with minor differences in the natural amino acid sequence. These modified insulins may have faster onset of action and/or shorter duration of action;

(j) Small molecule mimics of insulin, including, but not limited to L-783281, TE-17411, and the like;

(k) Na-glucose co-transporter inhibitors which inhibit the renal reabsorption of glucose such as T-1095, T-1095A, phlorizen, and the like;

(l) Amylin agonists which include, but are not limited to pramlintide, and the like; and

(k) Glucagon antagonists such as AY-279955, and the like.

As used herein, unless otherwise noted, the term “anti-lipid agent” shall mean any pharmaceutical agent capable of lowering triglycerides, lowering lipids, elevating HDL levels or improving the triglyceride/HDL Cholesterol ratio. Suitable examples include, but are not limited to, anti-lipemic agents, bile acid resins, cholesterol absorption inhibitors, fibric acid derivatives, HMG-CoA reductase inhibitors (i.e. statins). Preferable, the anti-lipid agent is a statin selected from the group consisting of atorvastatin (Lipitor), cerivastatin (Baycol), fluvastatin (Lescol), lovastatin, (Mevacor), pravastatin (Pravachol), rosuvastatin (Crestor), simvastatin (Zocor).

As used herein, unless otherwise noted, the term “anti-obesity agent” shall mean any pharmaceutical agent that treats obesity, promotes weight loss and/or suppresses appetite. Suitable examples of weight loss promoting include, but are not limited to rimonabant, orlistat, sibutramine, mazindol, benzphetamine, phenmetrazine, phentermine, diethylpropion, mazindol, phenylpropanolamine, ephedrine, quipazine, fluoxetine, sertraline, fenfluramine, dexfenfluramine, apomorphine, Exendin, dehydroepiandrosterone, etiocholandione, testosterone, oxandrolone, topiramate, and the like. Preferably, the weight loss promoting agent is rimonabant, topiramate, orlistat or sibutramine.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Wherein the present invention is directed to co-therapy or combination therapy, comprising administration of one or more compound(s) of formula (I) and one or more anti-diabetic and/or anti-lipid agents, “therapeutically effective amount” shall mean that amount of the combination of agents taken together so that the combined effect elicits the desired biological or medicinal response. For example, the therapeutically effective amount of co-therapy comprising administration of a compound of formula (I) and the anti-diabetic and/or anti-lipid agent would be the amount of the compound of formula (I) and the amount of the antidepressant that when taken together or sequentially have a combined effect that is therapeutically effective. Further, it will be recognized by one skilled in the art that in the case of co-therapy with a therapeutically effective amount, as in the example above, the amount of the compound of formula (I) and/or the amount of the anti-diabetic and/or anti-lipid agent individually may or may not be therapeutically effective.

As used herein, the terms “co-therapy” and “combination therapy” shall mean treatment of a subject in need thereof by administering one or more compounds of formula (I) in combination with one or more anti-diabetic and/or anti-lipid agent(s), wherein the compound(s) of formula (I) and the anti-diabetic and/or anti-lipid agent(s) are administered by any suitable means, simultaneously, sequentially, separately or in a single pharmaceutical formulation. Where the compound(s) of formula (I) and the anti-diabetic and/or anti-lipid agent(s) are administered in separate dosage forms, the number of dosages administered per day for each compound may be the same or different. The compound(s) of formula (I) and the anti-diabetic and/or anti-lipid agent(s) may be administered via the same or different routes of administration. Examples of suitable methods of administration include, but are not limited to, oral, intravenous (iv), intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of formula (I) and the anti-diabetic and/or anti-lipid agent(s) may be administered according to simultaneous or alternating regimens, at the same or different times during the course of the therapy, concurrently in divided or single forms.

In an embodiment of the present invention, the compound of formula (I) is selected from the group wherein

R¹ is selected from the group consisting of hydrogen, halogen, hydroxy, methoxy, trifluoromethyl, nitro and cyano;

X-Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is selected from the group consisting of hydrogen and methyl;

R³ and R⁴ are each independently selected from the group consisting of hydrogen and methyl;

alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;

or a pharmaceutically acceptable salt thereof.

In another embodiment of the present invention, the compound of formula (I) is selected from the group wherein

R¹ is selected from the group consisting of hydrogen and halogen;

X-Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is selected from the group consisting of hydrogen and methyl;

R³ and R⁴ are each independently selected from the group consisting of hydrogen and methyl;

and pharmaceutically acceptable salts thereof.

In another embodiment of the present invention, the compound of formula (I) is selected from the group wherein

R¹ is selected from the group consisting of hydrogen and halogen; wherein the halogen is bound at the 4-, 5- or 7-position;

X-Y is selected from the groups consisting of —O—CH—, —O—C(CH₃)—, —S—CH—, —S—C(CH₃)—, —N(CH₃)—CH— and —CH═CH—CH—;

A is selected from the group consisting of —CH₂— and —CH(CH₃)—;

R² is hydrogen;

R³ and R⁴ are each hydrogen;

and pharmaceutically acceptable salts thereof.

In another embodiment of the present invention, the compound of formula (I) is selected from the group wherein

R¹ is hydrogen;

X-Y is selected from the groups consisting of —O—CH—, —O—C(CH₃)—, —S—CH—, —S—C(CH₃)—, —N(CH₃)—CH— and —CH═CH—CH—;

A is selected from the group consisting of —CH₂— and —CH(CH₃)—;

R² is hydrogen;

R³ and R⁴ are each hydrogen;

and pharmaceutically acceptable salts thereof.

In another embodiment of the present invention, the compound of formula (I) is selected from the group wherein

R¹ is selected from the group consisting of hydrogen halogen, hydroxy, methoxy, trifluoromethyl, nitro and cyano; preferably, R¹ is selected from the group consisting of hydrogen and halogen; more preferably, R¹ is selected from the group consisting of hydrogen and halogen, wherein the halogen is bound at the 4-, 5- or 7-position;

X-Y is —S—CH—;

A is selected from the group consisting of —CH₂— and —CH(CH₃)—;

R² is selected from the group consisting of hydrogen and methyl; preferably, R² is hydrogen;

R³ and R⁴ are each independently selected from the group consisting of hydrogen and halogen; preferably, R³ and R⁴ are each hydrogen;

and pharmaceutically acceptable salts thereof.

In an embodiment of the present invention R¹ is selected from the group consisting of hydrogen, chloro, fluoro and bromo. In another embodiment of the present invention, the R¹ group is other than hydrogen and bound at the 4-, 5- or 7-position, preferably at the 5-position. In yet another embodiment of the present invention, the R¹ group is other than hydrogen and bound at the 5-, 6- or 8-position, preferably at the 6-position. In yet another embodiment of the present invention, R¹ is selected from the group consisting of hydrogen and halogen. In yet another embodiment of the present invention, R¹ is selected from the group consisting of hydroxy and methoxy. In yet another embodiment of the present invention, R¹ is selected from the group consisting of hydrogen, halogen and trifluoromethyl. In yet another embodiment of the present invention, R¹ is selected from the group consisting of hydrogen, halogen, trifluoromethyl, cyano and nitro. In yet another embodiment of the present invention, R¹ is selected from the group consisting of hydrogen, halogen, trifluoromethyl and cyano. In yet another embodiment of the present invention, R¹ is selected from the group consisting of trifluoromethyl and cyano. In yet another embodiment of the present invention, R¹ is selected from the group consisting of hydrogen, 4-bromo, 5-chloro, 5-fluoro, 5-bromo, 5-trifluoromethyl-5-cyano and 7-cyano.

In an embodiment of the present invention R² is hydrogen. In another embodiment of the present invention R³ and R⁴ are each hydrogen. In yet another embodiment of the present invention R² is hydrogen, R³ is hydrogen and R⁴ is hydrogen.

In an embodiment of the present invention, R³ and R⁴ are each independently selected from the group consisting of hydrogen and C₁₋₄alkyl. In another embodiment of the present invention, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S.

In an embodiment of the present invention, R³ and R⁴ are each independently selected from the group consisting of hydrogen, methyl and ethyl. In another embodiment of the present invention, R³ and R⁴ are each independently selected from the group consisting of hydrogen and methyl. In yet another embodiment of the present invention, R³ and R⁴ are each independently selected from the group consisting of hydrogen and ethyl. In yet another embodiment of the present invention, R³ is hydrogen and R⁴ is ethyl.

In an embodiment of the present invention R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, S and N. In another embodiment of the present invention R³ and R⁴ are taken together with the nitrogen atom to which they are bound to form a 5 to 7 membered saturated ring structure, optionally containing one to two additional heteroatoms independently selected from the group consisting of O, S and N. In another embodiment of the present invention R³ and R⁴ are taken together with the nitrogen atom to which they are bound to form a 5 to 7 membered aromatic ring structure, optionally containing one to two additional heteroatoms independently selected from the group consisting of O, S and N.

Preferably, R³ and R⁴ are taken together with the nitrogen atom to which they are bound to form a 5 to 6 membered saturated, partially unsaturated or aromatic ring structure, optionally containing one to two additional heteroatoms independently selected from the group consisting of O, S and N. More preferably, R³ and R⁴ are taken together with the nitrogen atom to which they are bound to form a 6 membered saturated, partially unsaturated or aromatic ring structure, optionally containing one to two additional heteroatoms independently selected from the group consisting of O, S and N.

Preferably, R³ and R⁴ are taken together with the nitrogen atom to which they are bound to form a 5 to 7 (more preferably 5 to 6) membered saturated or aromatic ring structure, optionally containing one to two (preferably one) additional heteroatoms independently selected from the group consisting of O, S and N (preferably O or N, more preferably N).

In another embodiment of the present invention, R³ and R⁴ are taken together with the nitrogen atom to which they are bound to form a 5 to 6 membered saturated or aromatic ring structure, optionally containing one to two (preferably one) additional heteroatoms independently selected from the group consisting of O, S and N (preferably O or N, more preferably, N).

Preferably, the 5 to 7 membered saturated, partially unsaturated or aromatic ring structure contains 0 to 1 additional heteroatoms independently selected from the group consisting of O, S and N. Preferably, the heteroatom is independently selected from the group consisting of O and N, more preferably, the heteroatom is N.

Suitable examples of the 5 to 7 membered, saturated, partially unsaturated or aromatic ring structures which optionally contain one to two additional heteroatoms independently selected from the group consisting of O, S and N include, but are not limited to pyrrolyl, pyrrolidinyl, pyrrolinyl, morpholinyl, piperidinyl, piperazinyl, imidazolyl, pyrazolyl, pyridyl, imidazolyl, thiomorpholinyl, pyrazinyl, triazinyl, azepinyl, and the like. Preferred 5 to 7 membered, saturated, partially unsaturated or aromatic ring structures which optional containing one to two additional heteroatoms independently selected from the group consisting of O, S and N include, but are not limited, to imidazolyl, pyrrolidinyl, piperidinyl and morpholinyl.

In an embodiment of the present invention A is —CH₂—.

In an embodiment of the present invention X-Y is selected from the group consisting of —S—CH—, —O—CH—, —O—C(CH₃)—, —N(CH₃)—CH— and —CH═CH—CH—. In another embodiment of the present invention X-Y is selected from the group consisting of —S—CH—, —O—CH—, —O—C(CH₃)— and —CH═CH—CH—. In yet another embodiment of the present invention X-Y is selected form the group consisting of —S—CH—, —O—CH—, —O—C(CH₃)— and —N(CH₃)—CH—. In yet another embodiment of the present invention X-Y is selected from the group consisting of —S—CH—, —O—CH—, —N(CH₃)—CH— and —CH═CH—CH—. In yet another embodiment of the present invention X-Y is selected from the group consisting of —S—CH—, —O—CH— and —CH═CH—C—. In yet another embodiment of the present invention, X-Y is selected from the group consisting of —S—CH— and —O—CH—. In yet another embodiment of the present invention, X-Y is selected from the group consisting of S—CH—, —S—C(CH₃)—, —O—CH—, —O—C(CH₃)— and —N(CH₃)—CH—.

In an embodiment of the present invention, X— is —S—CH—. In another embodiment of the present invention X-Y is —CH═CH═CH—. In yet another embodiment of the present invention X-Y is —N(CH₃)—CH—. In yet another embodiment of the present invention X-Y is selected from the group consisting of —O—CH— and —O—C(CH₃)—.

In an embodiment, the present invention is directed to a compounds selected from the group consisting of N-(benzo[b]thien-3-ylmethyl)-sulfamide; N-[(5-chlorobenzo[b]thien-3-yl)methyl]-sulfamide; N-(3-benzofuranyl methyl)-sulfamide; N-[(5-fluorobenzo[b]thien-3-yl)methyl]-sulfamide; N-(1-benzo[b]thien-3-ylethyl)-sulfamide; N-(1-naphthalenylmethyl)-sulfamide; N-[(2-methyl-3-benzofuranyl)methyl]-sulfamide; N-[(5-bromobenzo[b]thien-3-yl)methyl]-sulfamide; N-[(4-bromobenzo[b]thien-3-yl)methyl]-sulfamide; N-[(7-fluorobenzo[b]thien-3-yl)methyl]-sulfamide; N-[(1-methyl-1H-indol-3-yl)methyl]-sulfamide; N-[(4-trifluoromethylbenzo[b]thien-3-yl)methyl]-sulfamide; N-[(4-cyanobenzo[b]thien-3-yl)methyl]-sulfamide; N-[(benzo[b]thien-3-yl)methyl]-sulfamoylpyrrolidine; N-[(benzo[b]thien-3-yl)methyl]-N′-ethylsulfamide; Imidazole-1-sulfonic acid [(benzo[b]thien-3-yl)methyl]-amide; and pharmaceutically acceptable salts thereof.

Additional embodiments of the present invention, include those wherein the substituents selected for one or more of the variables defined herein (i.e. R¹, R², R³, R⁴, X-Y and A) are independently selected to be any individual substituent or any subset of substituents selected from the complete list as defined herein.

Representative compounds useful in the methods of the present invention are as listed in Table 1 and 2, below.

TABLE 1
Representative Compounds of Formula (I)

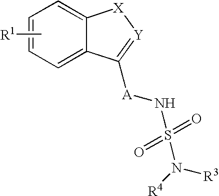

| ID No. | R¹ | —X—Y— | A | R³ | R⁴ |
|---|---|---|---|---|---|
| 1 | H | —S—CH— | —CH₂— | H | H |
| 3 | 5-Cl | —S—CH— | —CH₂— | H | H |
| 6 | H | —O—CH— | —CH₂— | H | H |
| 7 | H | —N(CH₃)—CH— | —CH₂— | H | H |
| 8 | 5-F | —S—CH— | —CH₂— | H | H |
| 9 | H | —S—CH— | —CH(CH₃)— | H | H |
| 10 | H | —CH═CH—CH— | —CH₂— | H | H |
| 13 | H | —O—C(CH₃) | —CH₂— | H | H |
| 15 | 5-Br | —S—CH— | —CH₂— | H | H |
| 17 | 4-Br | —S—CH— | —CH₂— | H | H |
| 18 | 7-F | —S—CH— | —CH₂— | H | H |
| 19 | 5-CF₃ | —S—CH— | —CH₂— | H | H |
| 20 | 5-CN | —S—CH— | —CH₂— | H | H |
| 21 | H | —S—CH— | —CH₂— | H | ethyl |

TABLE 2

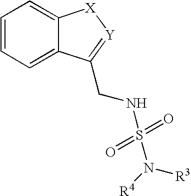

| ID No. | —X—Y— | R3 + R4 together with the N atom |
|---|---|---|
| 101 | —S—CH— | N-pyrrolidinyl |
| 102 | —S—CH— | N-imidazolyl |

As used herein, “halogen” shall mean chlorine, bromine, fluorine and iodine.

As used herein, the term “alkyl” whether used alone or as part of a substituent group, include straight and branched chains. For example, alkyl radicals include methyl, ethyl, propyl, isopropyl, butyl, isobutyl, sec-butyl, t-butyl, pentyl and the like. Unless otherwise noted, “C₁₋₄alkyl” means a carbon chain composition of 1-4 carbon atoms.

When a particular group is “substituted” (e.g., alkyl, phenyl, aryl, heteroalkyl, heteroaryl), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

Unless otherwise noted, the position at which the R¹ substituent is bound will be determined by counting around the core structure in a clockwise manner beginning at the X-Y positions as 1, 2 and continuing from thereon as follows:

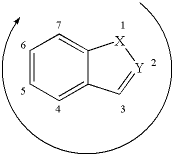

Should the X-Y substituent be —CH═CH—CH—, then the X-Y group will be counted as 1, 2, 3 and counting then continued clockwise around the core structure as previously noted.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

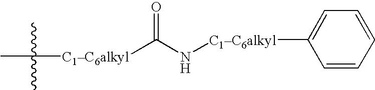

Abbreviations used in the specification, particularly the Schemes and Examples, are as follows:

DCE=Dichloroethane

DCM=Dichloromethane

DMF=N,N-Dimethylformamide

DMSO=Dimethylsulfoxide

LAH=Lithium Aluminum Hydride

MTBE=Methyl-tert-butyl ether

THF=Tetrahydrofuran

TLC=Thin Layer Chromatography

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following:

acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following:

acids including acetic acid, 2,2-dichlorolactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and

bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Compounds of formula (I) wherein A is —CH₂— may be prepared according to the process outlined in Scheme 1.

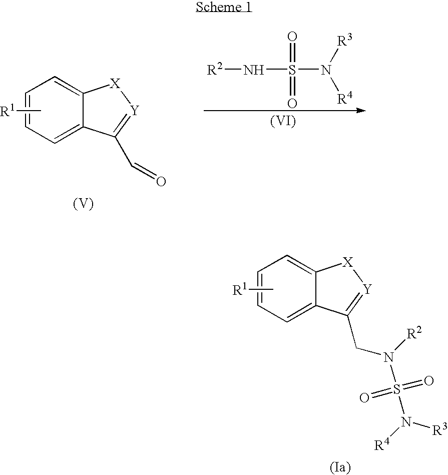

Accordingly, a suitably substituted compound of formula (V), a known compound or compound prepared by known methods, is reacted with a suitably substituted compound of formula (VI), a known compound or compound prepared by known methods, wherein the compound of formula (VI) is present in an amount in the range of about 2 to about 5 equivalents, in an organic solvent such as ethanol, methanol, dioxane, and the like, preferably, in an anhydrous organic solvent, preferably, at an elevated temperature in the range of about 50° C. to about 100° C., more preferably at about reflux temperature, to yield the corresponding compound of formula (Ia).

Compounds of formula (I) may alternatively be prepared according to the process outlined in Scheme 2.

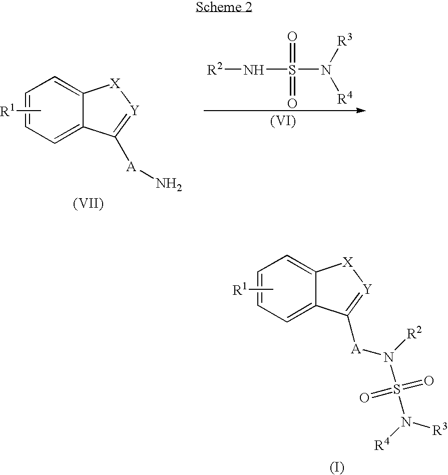

Accordingly, a suitably substituted compound of formula (VII), a known compound or compound prepared by known methods, is reacted with a suitably substituted compound of formula (VI), a known compound or compound prepared by known methods, wherein the compound of formula (VI) is present in an amount in the range of about 2 to about 5 equivalents, in an organic solvent such as THF, dioxane, and the like, preferably, in an anhydrous organic solvent, preferably, at an elevated temperature in the range of about 50° C. to about 100° C., more preferably at about reflux temperature, to yield the corresponding compound of formula (I).

Compounds of formula (VII) wherein A is —CH₂— may, for example, be prepared by according to the process outlined in Scheme 3.

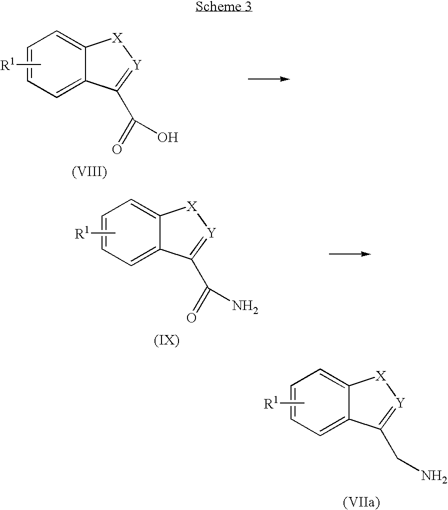

Accordingly, a suitably substituted a compound of formula (VIII), a known compound or compound prepared by known methods is reacted with an activating agent such as oxalyl chloride, sulfonyl chloride, and the like, and then reacted with an amine source such as ammonia, ammonium hydroxide, and the like, in an organic solvent such as THF, diethyl ether, DCM, DCE, and the like, to yield the corresponding compound of formula (IX).

The compound of formula (IX) is reacted with a suitably selected reducing agent such as LAH, borane, and the like, in an organic solvent such as THF, diethyl ether, and the like, to yield the corresponding compound of formula (VIIa).

Compounds of formula (VII) wherein A is —CH(CH₃)— may, for example, be prepared according to the process outlined in Scheme 4.

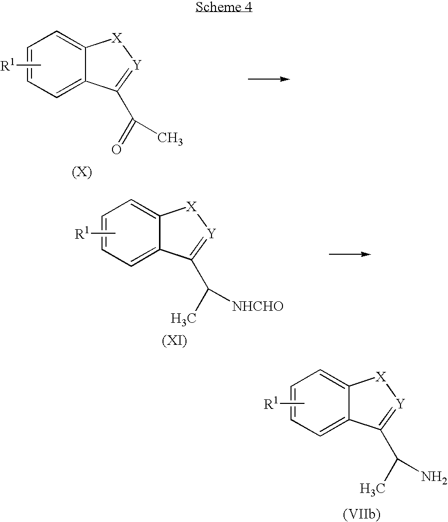

Accordingly, a suitably substituted compounds of formula (X), a known compound or compound prepared by known methods, is reacted with a mixture of formamide and formic acid, wherein the mixture of formamide and formic acid is present in an amount greater than about 1 equivalent, preferably, in an excess amount of greater than about 5 equivalent, at an elevated temperature of about 150° C., to yield the corresponding compound of formula (XI).

The compound of formula (XI) is hydrolyzed by reacting with concentrated HCl, concentrated H₂SO₄, and the like, at an elevated temperature, preferably at reflux temperature, to yield the corresponding compound of formula (VIIb).

Compounds of formula (VII) may alternatively, be prepared according to the process outlined in Scheme 5.

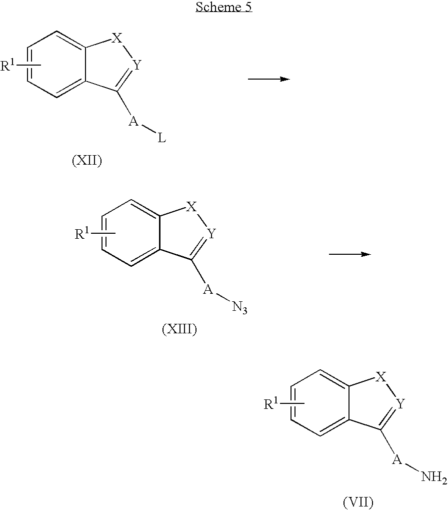

Accordingly, a suitably substituted compound of formula (XII), wherein L is a leaving group such as Br, Cl, I, tosylate, mesylate, and the like, a known compound or compound prepared by known methods, is reacted with sodium azide, in an organic solvent such a DMF, DMSO, methanol, ethanol, and the like, to yield the corresponding compound of formula (XIII).

The compound of formula (XIII) is reacted with a suitably selected reducing agent such as LAH, triphenylphosphine, H_{2(g)}, and the like, according to known methods, to yield the corresponding compound of formula (VII).

Compounds of formula (VII) wherein A is CH₂ and X-Y is —O—CH₂— may, for example, be prepared according to the process outlined in Scheme 6.

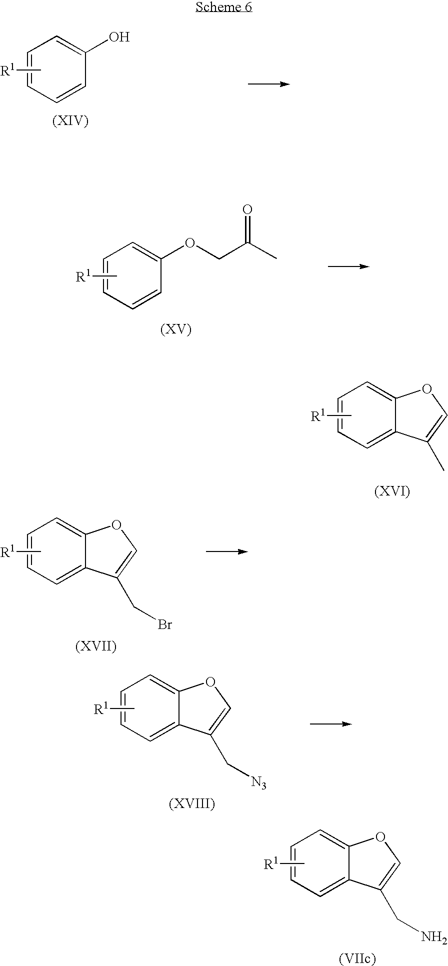

Accordingly, a suitably substituted phenol, a compound of formula (XIV), a known compound or compound prepared by known methods is reacted with bromoacetone, a known compound, in the presence of a base such as K₂CO₃, Na₂CO₃, NaH, triethylamine, pyridine, and the like, in an organic solvent such as acetonitrile, DMF, THF, and the like, optionally at an elevated temperature, to yield the corresponding compound of formula (XV).

The compound of formula (XV) is reacted with an acid such as polyphosphoric acid, sulfuric acid, hydrochloric acid, and the like, preferably with polyphosphoric acid, preferably in the absence of a solvent (one skilled in the art will recognize that the polyphosphoric acid acts as the solvent), to yield the corresponding compound of formula (XVI).

The compound of formula (XVI) is reacted with a source of bromine such as N-bromosuccinimide in the presence of benzoylperoixde, Br₂, and the like, in an organic solvent such as carbon tetrachloride, chloroform, DCM, and the like, preferably in a halogenated organic solvent, to yield the corresponding compound of formula (XVII).

The compound of formula (XVII) is reacted with sodium azide, in an organic solvent such a DMF, DMSO, methanol, ethanol, and the like, to yield the corresponding compound of formula (XVIII).

The compound of formula (XVIII) is reacted with a suitably selected reducing agent such as LAH, triphenylphosphine, H_{2(g)}, and the like, according to known methods, to yield the corresponding compound of formula (VIIc).

Compounds of formula (V) wherein X-Y is —S—CH— may, for example, be prepared according to the process outlined in Scheme 7.

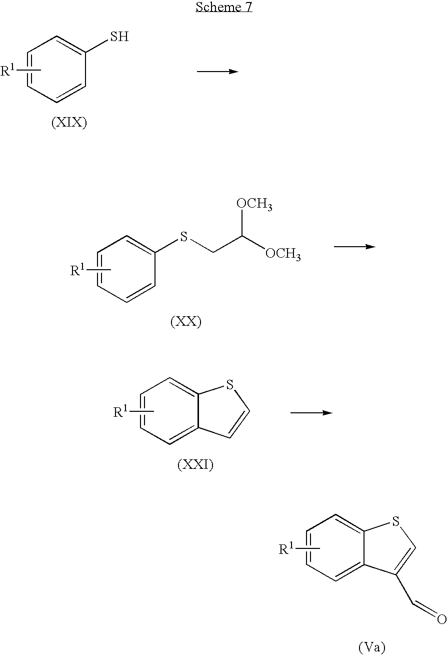

Accordingly, a suitably substituted compound of formula (XIX), a known compound or compound prepared by known methods is reacted with choroacetaldehyde dimethyl acetal or bromoacetaldehyde dimethyl acetal, a known compound, in the presence of a base such as potassium-tert-butoxide, sodium-tert-butxide, potassium carbonate, potassium hydroxide, and the like, in an organic solvent such as THF, DMF, acetonitrile, and the like, to yield the corresponding compound of formula (XX).

The compound of formula (XX) is reacted with reacted with an acid such as polyphosphoric acid, sulfuric acid, hydrochloric acid, and the like, preferably with polyphosphoric acid in the presence of chlorobenzene, preferably in the absence of a solvent (one skilled in the art will recognize that the polyphosphoric acid and/or the chlorobenzene may act as the solvent), at an elevated temperature in the range of from about 100 to 200° C., preferably at an elevated temperature of about reflux temperature, to yield the corresponding compound of formula (XXI).

The compound of formula (XXI) is reacted with a formylating reagent such as dichloromethyl methyl ether, and the like, in the presence of Lewis acid catalyst such as titanium tetrachloride, aluminum trichloride, tin tetrachloride, and the like, in an organic solvent such as DCM, chloroform, and the like, at a temperature in the range of from about 0° C. to about room temperature, to yield the corresponding compound of formula (Va).

Compounds of formula (I) wherein R³ and/or R⁴ are other than hydrogen or R³ and R⁴ are taken together with the nitrogen to which they are bound to form a ring structure, may alternatively be prepared according to the process outlined in Scheme 8.

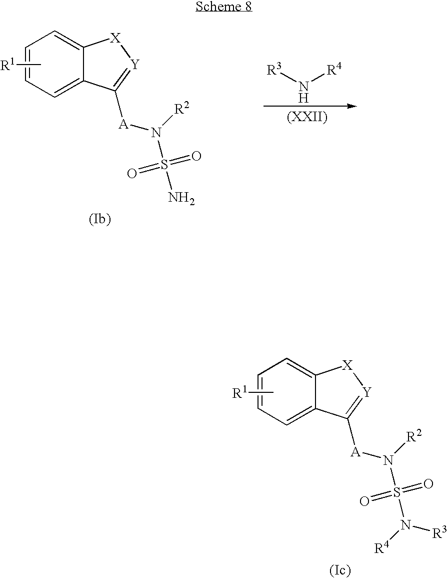

Accordingly, a suitably substituted compound of formula (Ib), is reacted with a suitably substituted amine, a compound of formula (XXII), a known compound or compound prepared by known methods, in water or an organic solvent such as dioxane, ethanol, THF, isopropanol, and the like, provide that the compound of formula (Ib) and the compound of formula (XXII) are at least partially soluble in the water or organic solvent, at a temperature in the range of from about room temperature to about reflux, preferably at about reflux temperature, to yield the corresponding compound of formula (Ic).

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography.

The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.1-1000 mg and may be given at a dosage of from about 0.01-200.0 mg/kg/day, preferably from about 0.1 to 100 mg/kg/day, more preferably from about 0.5-50 mg/kg/day, more preferably from about 1.0-25.0 mg/kg/day, more preferably from about 0.5-10.0 mg/kg/day, most preferably from about 1.0 to about 5.0 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 1000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of the present invention may also be carried out using a pharmaceutical composition comprising any of the compounds as defined herein and a pharmaceutically acceptable carrier. The pharmaceutical composition may contain between about 0.1 mg and 1000 mg, preferably about 50 to 500 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Compounds of this invention may be administered in any of the foregoing compositions and according to dosage regimens established in the art whenever treatment of glucose and/or lipid related disorders is required.

The daily dosage of the products may be varied over a wide range from 0.01 to 200 mg/kg per adult human per day or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 200 mg/kg of body weight per day. Preferably, the range is from about 0.1 to about 100.0 mg/kg of body weight per day, more preferably, from about 0.5 mg/kg to about 50 mg/kg, more preferably, from about 1.0 to about 25.0 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

### EXAMPLE 1

### N-(benzo[b]thien-3-ylmethyl)-sulfamide (Compound #1)

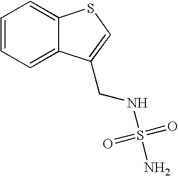

Thianaphthene-3-carboxaldehyde (1.62 g, 10.0 mmol) was dissolved in anhydrous ethanol (50 mL). Sulfamide (4.0 g, 42 mmol) was added and the mixture was heated to reflux for 16 hours. The mixture was cooled to room temperature. Sodium borohydride (0.416 g, 11.0 mmol) was added and the mixture was stirred at room temperature for three hours. The reaction was diluted with water (50 mL) and extracted with chloroform (3×75 mL). The extracts were concentrated and chromatographed (5% methanol in DCM) to yield the title compound as a white solid.

¹H NMR (DMSO-d₆): δ 7.98 (1H, dd, J=6.5, 2.3 Hz), 7.92 (1H, dd, J=6.6, 2.4 Hz), 7.62 (1H, s), 7.36-7.45 (2H, m), 7.08 (1H, t, J=6.3 Hz), 6.72 (2H, s), 4.31 (2H, d, J=6.3 Hz).

### EXAMPLE 2

### N-[(5-chlorobenzo[b]thien-3-yl)methyl]-sulfamide (Compound #3)

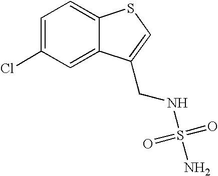

(5-Chloro-1-benzothiophene-3-yl)methylamine (0.820 g, 4.15 mmol) and sulfamide (2.5 g, 26 mmol) were combined in anhydrous dioxane (50 mL) and the mixture was heated to reflux for four hours. The reaction was cooled and diluted with water (50 mL). The solution was extracted with chloroform (3×75 mL). The extracts were concentrated and chromatographed (5% methanol in DCM) to yield the title compound as a white solid.

¹H NMR (DMSO-d₆): δ 8.05 (2H, m), 7.74 (1H, s), 7.40 (1H, d, J=6.5 Hz), 7.07 (1H, t, J=6.3 Hz), 6.72 (2H, s), 4.26 (2H, d, J=6.4 Hz).

### EXAMPLE 3

### N-[(1-methyl-1H-indol-3-yl)methyl]-sulfamide (Compound #7)

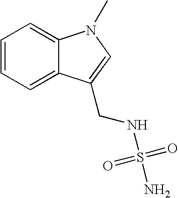

N-Methylindole-3-carboxaldehyde (1.66 g, 10.4 mmol) was dissolved in anhydrous ethanol (50 mL). Sulfamide (4.5 g, 47 mmol) was added and the mixture was heated to reflux for 16 hours. Additional sulfamide (1.0 g, 10.4 mmol) was added and the mixture was heated to reflux for 24 hours. The mixture was cooled to room temperature. Sodium borohydride (0.722 g, 12.5 mmol) was added and the mixture was stirred at room temperature for one hour. The reaction was diluted with water (50 mL) and extracted with DCM (3×75 mL). The extracts were concentrated and about 1 mL of methanol was added to create a slurry which was filtered to yield the title compound as a white powder.

¹H NMR (CD₃OD): δ 7.67 (1H, d, J=5.9 Hz), 7.32 (1H, d, J=6.2 Hz), 7.14-7.19 (2H, m), 7.06 (1H, dt, J=7.7, 0.7 Hz), 4.36 (2H, s), 3.75 (3H, s)

MS (M−H)⁻ 237.6.

### EXAMPLE 4

### N-(3-benzofuranylmethyl)-sulfamide (Compound #6)

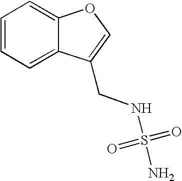

Benzofuran-3-carboxylic acid (1.91 g, 11.8 mmol) was suspended in anhydrous DCM (75 mL). Oxalyl chloride (2.0 M in DCM, 6.48 mL) and then one drop of dimethylformamide were added. The solution was stirred at room temperature for two hours, then ammonium hydroxide (concentrated, 10 mL) was added. The resulting mixture was diluted with water (100 mL) and extracted with DCM (3×100 mL). The extracts were concentrated to a gray solid and dissolved in anhydrous THF (100 mL). Lithium aluminum hydride (1.0 M in THF, 11.8 mL) was added. The mixture was stirred at room temperature for 16 hours. A minimal amount of saturated aqueous NaHCO₃ and then MgSO₄ were added. The mixture was filtered and then extracted with 1 N HCl. The aqueous extracts were adjusted to pH 14 with 3N NaOH and extracted with DCM. The organic extracts were dried with magnesium sulfate and concentrated to a colorless oil. The oil was dissolved in dioxane (50 mL) and sulfamide (3.7 g, 38 mmol) was added. The mixture was heated to reflux for 4 hours, cooled to room temperature, and concentrated. The resulting solid was chromatographed (5% methanol in DCM) to yield the title compound as a slightly yellow solid.

¹H NMR (CD₃OD): δ 7.53 (1H, d, J=5.7 Hz), 7.44 (1H, d, J=6.0 Hz), 7.16-7.26 (2H, m), 6.73 (1H, s), 4.35 (2H, s).

### EXAMPLE 5

### N-[(5-fluorobenzo[b]thien-3-yl)methyl]-sulfamide (Compound #8)

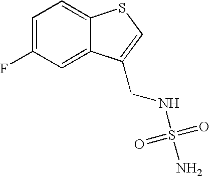

5-Fluoro-3-methylbenzothiophene (1.14 g, 6.83 mmol), benzoyl peroxide (0.165 g, 0.68 mmol) and N-bromosuccinimide (1.70 g, 7.52 mmol) were combined in carbon tetrachloride (25 mL) and the mixture was heated to reflux for 3 hours. The yellow solution was cooled, diluted with water, and extracted with DCM (2×50 mL). The extracts were washed with brine (100 mL), dried with magnesium sulfate, and concentrated to an orange solid. The solid was dissolved in anhydrous DMF. Sodium azide (4.0 g, 61 mmol) was added and the mixture was stirred for 16 hours at room temperature. The reaction was diluted with water (100 mL) and extracted with diethyl ether (2×75 mL). The extracts were washed with brine (100 mL), dried with magnesium sulfate, and concentrated to a yellow oil. The oil was dissolved in a mixture of THF (50 mL) and water (5 mL). Triphenylphosphine (3.60 g, 13.7 mmol) was added. The mixture was stirred at room temperature for 16 hours. The reaction was concentrated and chromatographed (2 to 5% methanol in DCM). The resulting C-(5-fluoro-benzo[b]thien-3-yl)-methylamine (1.04 g, 5.73 mmol) was dissolved in anhydrous dioxane (50 mL) and sulfamide (2.75 g, 28.7 mmol) was added. The reaction was heated to reflux for 4 hours, cooled to room temperature, and concentrated to a solid which was chromatographed (5% methanol in DCM) to yield the title compound as a white solid.

¹H NMR (CD₃OD): δ 7.85 (1H, dd, J=6.6, 3.6 Hz), 7.66 (1H, dd, J=7.4, 1.8 Hz), 7.62 (1H, s), 7.13-7.18 (1H, m), 4.40 (2H, s).

### EXAMPLE 6

### N-(1-benzo[b]thien-3-ylethyl)-sulfamide (Compound #9)

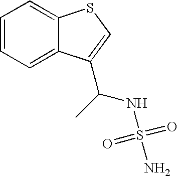

3-Acetylthianaphthene (3.00 g, 17.0 mmol) was added to a mixture of formic acid (10 mL) and formamide (10 mL). The solution was heated to 150° C. for 8 hours. The reaction was cooled to room temperature, diluted with water (50 mL), and extracted with diethyl ether (3×50 mL). The ether extracts were washed with saturated aqueous NaHCO₃ and brine. The solution was concentrated and chromatographed (5% methanol in DCM) to yield N-(1-benzo[b]thiophen-3-yl-ethyl)-formamide (1.76 g) as a white solid which was suspended in concentrated HCl (30 mL). The mixture was heated to reflux for 1.5 hours then diluted with water (100 mL). 3N NaOH was added until the pH was 14. The mixture was extracted with diethyl ether (3×100 mL) then dried with magnesium sulfate and concentrated to an orange oil. The oil was dissolved in anhydrous dioxane (75 mL) and sulfamide was added. The mixture was heated to reflux for 2 hours then diluted with water (50 ml). The solution was extracted with ethyl acetate (2×50 mL), dried with magnesium sulfate, concentrated, and chromatographed (2.5% to 5% methanol in DCM) to yield the title compound as a white solid.

¹H NMR (CD₃OD): δ 8.01 (1H, dd, J=5.5, 0.7 Hz), 7.85 (1H, dt, J=6.0, 0.6 Hz), 7.49 (1H, s), 7.31-7.40 (2H, m), 4.95 (1H, q, J=5.1 Hz), 1.67 (3H, d, J=5.1 Hz).

### EXAMPLE 7

### N-(1-naphthalenylmethyl)-sulfamide (Compound #10)

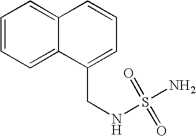

1-Naphthanlenemethylamine (2.00 g, 12.7 mmol) and sulfamide (5.0 g, 52 mmol) were combined in anhydrous dioxane (100 mL) and the mixture was heated to reflux for 6 hours. The reaction was cooled to room temperature and was filtered. The filtrate was concentrated to a solid and washed with water until TLC indicated no remaining trace of sulfamide in the solid. The collected solid was dried under vacuum to yield the title compound as a white solid.

¹H NMR (CDCl₃): δ 8.09 (1H, d, J=6.3 Hz), 7.86 (1H, dd, J=12.9, 6.2 Hz), 7.42-7.61 (4H, m), 4.75 (2H, d, J=4.4 Hz), 4.58 (1H, br s), 4.51 (2H, br s).

### EXAMPLE 8

### N-[(2-methyl-3-benzofuranyl)methyl]-sulfamide (Compound #13)

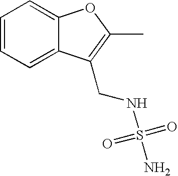

2-Methylbenzofuran-3-carbaldehyde (0.51 g, 3.18 mmol) was dissolved in anhydrous ethanol (25 mL). Sulfamide (1.5 g, 16 mmol) was added and the mixture was heated to reflux for 4 days. The mixture was cooled to room temperature. Sodium borohydride (0.132 g, 3.50 mmol) was added and the mixture was stirred at room temperature for 24 hours. The reaction was diluted with water (100 mL) and extracted with DCM (3×75 mL). The extracts were concentrated and suspended in a minimal amount of DCM and filtered to yield the title compound as a white solid.

¹H NMR (DMSO-d₆): δ 7.65 (1H, dd, J=6.4, 2.6 Hz), 7.43-7.47 (1H, m), 7.19-7.23 (2H, m), 6.87 (1H, t, J=6.2 Hz), 6.68 (2H, s), 4.11 (2H, d, J=6.2 Hz), 2.42 (3H, s).

### EXAMPLE 9

### N-[(5-bromobenzo[b]thien-3-yl)methyl]-sulfamide (Compound #15)

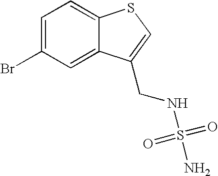

5-Bromobenzothiophene (1.60 g, 7.51 mmol) and dichloromethyl methyl ether (1.29 g, 11.3 mmol) were dissolved in anhydrous 1,2-dichloroethane (75 mL). Titanium tetrachloride (2.14 g, 11.3 mmol) was added, turning the solution dark. After one hour at room temperature, the reaction was poured into a mixture of saturated aqueous NaHCO₃ and ice. The mixture was stirred for about 30 minutes and then was extracted with DCM (2×100 mL). The extracts were concentrated and chromatographed (0 to 5% ethyl acetate in hexane) to yield 5-bromo-benzo[b]thiophene-3-carbaldehyde (1.32 g). The 5-bromobenzothiophene-3-carboxaldehyde (1.20 g, 4.98 mmol) and sulfamide (4.0 g, 42 mmol) were combined in anhydrous ethanol (25 mL) and heated to reflux for three days. The reaction was cooled to room temperature and sodium borohydride (0.207 g, 5.47 mmol) was added. After five hours, water (50 ml) was added and the solution was extracted with chloroform (3×50 mL). The extracts were concentrated, suspended in a minimal amount of DCM, and filtered to provide the title compound as a yellow solid.

¹H NMR (DMSO-d₆): δ 8.12 (1H, d, J=1.8 Hz), 7.97 (1H, d, J=8.6), 7.71 (1H, s), 7.52 (1H, dd, J=8.6, 1.9 Hz), 7.12 (1H, t, J=6.3 Hz), 6.72 (2H, s), 4.28 (2H, d, J=6.2 Hz).

### EXAMPLE 10

### N-[(4-bromobenzo[b]thien-3-yl)methyl]-sulfamide (Compound #17)

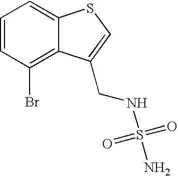

4-Bromobenzothiophene (1.80 g, 8.45 mmol) and dichloromethyl methyl ether (1.46 g, 12.7 mmol) were dissolved in anhydrous DCM (100 mL). Titanium tetrachloride (2.40 g, 12.7 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×150 mL). The extracts were concentrated and chromatographed (0 to 15% ethyl acetate in hexane) to yield 4-bromobenzothiophene-3-carboxaldehyde (0.910 g). The 4-bromobenzothiophene-3-carboxaldehyde (0.910 g, 3.77 mmol) and sulfamide (3.0 g, 31 mmol) were combined in anhydrous ethanol (25 mL) and heated to reflux for three days. The reaction was cooled to room temperature and sodium borohydride (0.157 g, 4.15 mmol) was added. After five hours, water (50 ml) was added and the solution was extracted with chloroform (3×50 mL). The extracts were concentrated, suspended in a minimal amount of DCM, and filtered to yield the title compound as a yellow solid.

¹H NMR (DMSO-d₆): δ 8.05 (1H, dd, J=8.1, 0.8 Hz), 7.78 (1H, s), 7.64 (1H, dd, J=7.6, 0.8 Hz), 7.27 (1H, t, J=7.9 Hz), 7.13 (1H, t, J=6.3 Hz), 6.72 (2H, br s), 4.65 (2H, d, J=5.3 Hz).

### EXAMPLE 11

### N-[(7-fluorobenzo[b]thien-3-yl)methyl]-sulfamide (Compound #18)

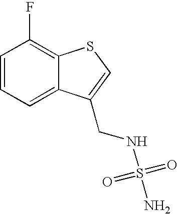

2-Fluorothiophenol (4.14 g, 32.6 mmol) was dissolved in anhydrous THF (100 mL). Potassium tert-butoxide (1.0 M in THF, 35.8 mL) was added and the suspension was stirred at room temperature for 15 minutes. 2-Chloroacetaldehyde dimethyl acetal was added and the mixture was stirred for 3 days. Water (100 mL) was added and the solution was extracted with diethyl ether (3×100 mL). The extracts were concentrated to a yellow oil and chromatographed (5 to 20% ethyl acetate in hexane) to yield 1-(2,2-dimethoxy-ethylsulfanyl)-2-fluoro-benzene (6.42 g) as a colorless oil. Chlorobenzene (25 mL) was heated to reflux and polyphosphoric acid (1 mL) was added. The 1-(2,2-dimethoxy-ethylsulfanyl)-2-fluoro-benzene was then added slowly turning the solution dark. After 3 hours of heating, the reaction was cooled to room temperature and diluted with water (50 mL). The solution was extracted with benzene (2×50 mL). The extracts were concentrated and chromatographed (0 to 15% ethyl acetate in hexane) to yield 7-fluorobenzothiophene (0.77 g). The 7-fluorobenzothiophene (0.77 g, 5.1 mmol) and dichloromethyl methyl ether (0.872 g, 7.6 mmol) were dissolved in anhydrous DCM (25 mL). Titanium tetrachloride (1.0 M in DCM, 7.6 mL, 7.6 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×50 mL). The extracts were concentrated and chromatographed (0 to 15% ethyl acetate in hexane) to yield 7-fluorobenzothiophene-3-carboxaldehyde (0.642 g). The 7-fluorobenzothiophene-3-carboxaldehyde (0.642 g, 3.77 mmol) and sulfamide (1.7 g, 18 mmol) were combined in anhydrous ethanol (20 mL) and heated to reflux for three days. The reaction was cooled to room temperature and sodium borohydride (0.148 g, 3.92 mmol) was added. After two hours, water (25 ml) was added and the solution was extracted with chloroform (3×25 mL). The extracts were concentrated, suspended in a minimal amount of DCM, and filtered to yield the title compound as a yellow solid.

¹H NMR (DMSO-d₆): δ 7.78 (1H, d, J=8.0 Hz), 7.43-7.50 (1H, m), 7.27 (1H, dd, J=10.3, 7.9 Hz), 7.14 (1H, t, J=6.4 Hz), 6.74 (2H, br s), 4.31 (2H, d, J=6.4 Hz).

### EXAMPLE 12

### N-[(4-trifluoromethylbenzo[b]thien-3-yl)methyl]-sulfamide (Compound #19)

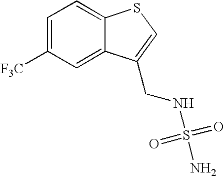

4-Trifluoromethylbenzothiophene (0.276 g, 1.37 mmol) and dichloromethyl methyl ether (0.236 g, 2.06 mmol) were dissolved in anhydrous DCM (10 mL). Titanium tetrachloride (1.0M in DCM, 2.1 mL, 2.1 mmol) was added, turning the solution dark. After 30 minutes at room temperature, the reaction was poured into a mixture of saturated aqueous NaHCO₃ and ice. The mixture was stirred for about 30 minutes and then extracted with DCM (2×25 mL). The extracts were concentrated and chromatographed (0 to 15% ethyl acetate in hexane) to yield 4-trifluoromethylbenzothiophene-3-carboxaldehyde.

The 4-trifluoromethylbenzothiophene-3-carboxaldehyde (0.226 g, 0.982 mmol) and sulfamide (0.471 g, 4.91 mmol) were combined in anhydrous ethanol (5 mL) and heated to reflux for 24 hours. The reaction was cooled to room temperature and sodium borohydride (0.056 g, 1.47 mmol) was added. After five hours, water (10 ml) was added and the solution was extracted with chloroform (3×10 mL). The extracts were concentrated, and chromatographed (5% methanol in DCM) to yield the title compound as a white solid.

¹H NMR (DMSO-d₆): δ 8.30 (1H, s), 8.25 (1H, d, J=8.4 Hz), 7.84 (1H, s), 7.68 (1H, dd, J=8.5, 1.4 Hz), 6.7-6.9 (2H, br s), 4.4-4.5 (1H, br s), 4.37 (2H, s).

### EXAMPLE 13

### N-[(4-cyanobenzo[b]thien-3-yl)methyl]-sulfamide (Compound #20)

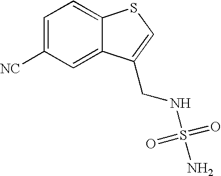

4-Cyanobenzothiophene (1.15 g, 7.22 mmol) and dichloromethyl methyl ether (1.25 g, 10.8 mmol) were dissolved in anhydrous DCM (100 mL). Titanium tetrachloride (1.0M in DCM, 10.8 mL, 10.8 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×50 mL). The extracts were concentrated and chromatographed (0 to 15% ethyl acetate in hexane) to yield 4-cyanobenzothiophene-3-carboxaldehyde.

The 4-cyanobenzothiophene-3-carboxaldehyde (0.298 g, 1.59 mmol) and sulfamide (0.766 g, 7.97 mmol) were combined in anhydrous ethanol (20 mL) and heated to reflux for 24 hours. The reaction was cooled to room temperature and sodium borohydride (0.091 g, 2.39 mmol) was added. After five hours, water (20 ml) was added and the solution was extracted with chloroform (3×20 mL). The extracts were concentrated, and chromatographed (5% methanol in DCM) to yield the title compound as a white solid.

¹H NMR (DMSO-d₆): δ 8.37 (1H, s), 8.30 (1H, d, J=8.4 Hz), 7.87 (1H, s), 7.70 (1H, dd, J=8.5, 1.4 Hz), 6.7-6.9 (2H, br s), 4.4-4.5 (1H, br s), 4.40 (2H, s).

### EXAMPLE 14

### N-[(benzo[b]thien-3-yl)methyl]-sulfamoylpyrrolidine (Compound #101)

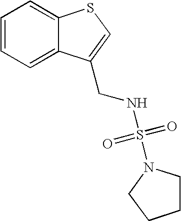

N-[(Benzo[b]thien-3-yl)methyl]-sulfamide (0.250 g, 1.03 mmol) and pyrrolidine (0.25 mL) were combined in anhydrous dioxane (5 mL) and heated to reflux for 32 hours. The reaction was evaporated and chromatographed with 5% methanol in DCM to yield the title compound as a white solid.

¹H NMR (CDCl₃): δ 7.84-7.89 (2H, m), 7.38-7.45 (3H, m), 4.49 (3H, br s), 3.25 (4H, t, J=4.0 Hz), 1.80 (4H, t, J=4.0 Hz).

### EXAMPLE 15

### N-[(benzo[b]thien-3-yl)methyl]-N′-ethylsulfamide (Compound #21)

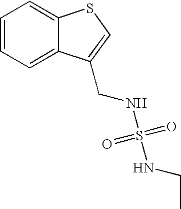

N-[(Benzo[b]thien-3-yl)methyl]-sulfamide (0.250 g, 1.03 mmol) and ethylamine (70% in H₂O, 0.10 mL) were combined in anhydrous dioxane (5 mL) and heated to reflux for 32 hours. The reaction was evaporated and chromatographed with 5% methanol in DCM to yield the title compound as a white solid.

¹H NMR (CDCl₃): δ 7.83-7.90 (2H, m), 7.36-7.47 (3H, m), 4.51 (2H, s), 2.90 (2H, q, J=7 Hz), 1.03 (3H, t, J=7 Hz).

### EXAMPLE 16

### Imidazole-1-sulfonic acid [(benzo[b]thien-3-yl)methyl]-amide (Compound #102)

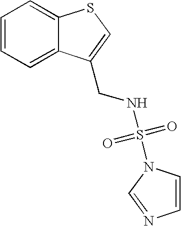

3-Benzothienylmethylamine and 3-(imidzole-1-sulfonyl)-1-methyl-3H-imidazol-1-ium triflate were combined in anhydrous acetonitrile. The solution was stirred at room temperature overnight, concentrated, and chromatographed (5% methanol in DCM) to yield the title compound as a tan solid.

¹H NMR (DMSO-d₆): δ 8.05 (1H, dd, J=7.0, 1.6 Hz), 7.99 (1H, dd, J=7.1, 1.7 Hz), 7.85 (1H, s), 7.66 (1H, s), 7.42-7.65 (5H, m), 4.34 (2H, s).

### EXAMPLE 17

### Diabetic db/db Mouse In Vivo Assay

Db/db mice are known in the art to be susceptible to Type II diabetes (Sharma K, McCue P, Dunn S R. Am J Physiol Renal Physiol. 2003 June; 284(6):F1138-44). The Db/db mice are also known in the art to be a useful model for dylipidemia.

Female db/db mice (C57BL/6J-Lep^{db/db}, Jackson Laboratories, Bar Harbor, Me., USA) were received at 8 weeks of age and single-housed and fed with regular chow diet. Blood was collected by tail puncture and glucose was monitored with a glucometer (OneTouch Basic, Lifescan, Newtown, Pa.).

Mice at 10 weeks of age were randomized into treatment groups based on glucose values (first criterion, average of 250 mg/dl) and body weight (second criterion, average of 37 gram). The mice were orally gavaged once daily (0.2 ml at 1500-1700 hour) with vehicle control (0.5% methylcellulose, pH7.4) and vehicle containing test compound (300 mg/kg). On day 11, the mice were fasted for 4 hr during light cycle (food was removed 0600-1000 hour) and blood glucose levels were measured through tail puncture with a glucometer at 1000 hour. The mice were then anaesthetized with sodium pentobarbital (1 ml/kg, i.p, Sleepaway, Fort Dodge, Iowa) and blood was drawn via cardiac puncture and collected into heparinized tubes.

White adipose tissue (WAT) (retroperitoneal fat) and skeletal muscle (gastrocnemius and soleus muscle) were dissected and weighed. Plasma samples were obtained by centrifuge at 2,000 g for 15 minutes at 4° C. and subjected to measurement of insulin, HDL cholesterol and triglyceride.

Data shown below are expressed as the mean and standard error calculated using 9-10 mice per treatment group. The 2 tailed Student's t-Tests were used for statistic analysis. All animal studies complied with the guideline of the Institutional Animal Care and Use Committee.

Compound #1 was evaluated according to the procedure described above. The blood glucose levels of female db/db mice were 255±15 mg/dl 5 days before the experiments. At the end of the experiment, the blood glucose levels of vehicle control mice were elevated 166% (420±22 mg/dl). The blood glucose levels in db/db mice were significantly lower with Compound #1 treatments as compared to vehicle treated mice. Insulin levels were increased in Compound #1 treated animals (14.6±3.0) versus vehicle treated animals (11.9±2.2).

Mice treated with Compound #1 also exhibited increased skeletal muscle mass. Further, mice treated with Compound #1 had a significant decrease in the fat to lean mass ratio.

Additionally, the plasma HDL cholesterol levels in db/db mice treated with Compound #1 were significantly increased, as compared to vehicle treated mice, whereas the blood triglyceride levels in db/db mice treated with Compound #1 were decreased as compared to vehicle treated mice.

A summary of the data for vehicle and Compound #1 treated mice measuring blood glucose levels, retroperitoneal fat, skeletal muscle mass, triglycerides and HDL cholesterol are as shown in Table 4, below.

TABLE 4
Diabetic db/db Mice In Vivo Results

| | Blood Glucose | Retroperitoneal Fat | Skeletal Muscle |
|---|---|---|---|
| | (mg/dl) | Weight (g) | Weight (g) |
| Vehicle | 420 ± 22 | 1.47 ± 0.05 | 0.136 ± 0.007 |
| Compound #1 | 350 ± 19 | 1.29 ± 0.09 | 0.155 ± 0.004 |
| | p < 0.05 | | p < 0.01 |

| | Triglycerides | HDL Cholesterol |
|---|---|---|
| | (mg/dl) | (mg/dl) |
| Vehicle | 161 ± 18.1 | 45.5 ± 1.3 |
| Compound #1 | 149.2 ± 15.8 | 52.6 ± 1.8 |
| | | p < 0.05 |

Thus, the data show that Compound #1 was effective at (a) lowering blood glucose levels, (b) lowering triglycerides and (c) elevating HDL cholesterol levels. Additionally animals treated with Compound #1 had more muscle mass than those treated with vehicle, which suggests that Compound #1 may preserve muscle mass i.e. prevent diabetic cachexia.

### EXAMPLE 18

As a specific embodiment of an oral composition, 100 mg of the Compound #1 prepared as in Example 1 is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A method for a glucose related disorder comprising administering to a subject in need thereof, a therapeutically effective amount of a compound of formula (I) 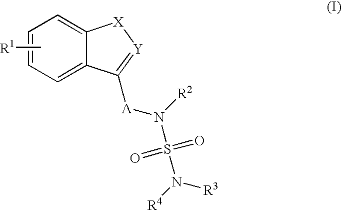
wherein
R¹ is selected from the group consisting of hydrogen, halogen, hydroxy, methoxy, trifluoromethyl, nitro and cyano;
X-Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

2. The method of claim 1 wherein
R¹ is selected from the group consisting of hydrogen, halogen, trifluoromethyl, cyano and nitro;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen, methyl and ethyl;
or a pharmaceutically acceptable salt thereof.

3. The method of claim 2, wherein
R¹ is selected from the group consisting of hydrogen, halogen, trifluoromethyl and cyano;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each independently selected from the group consisting of hydrogen and ethyl;
or a pharmaceutically acceptable salt thereof.

4. The method of claim 3, wherein
R¹ is selected from the group consisting of hydrogen, 5-chloro, 5-fluoro, 5-bromo, 4-bromo, 7-fluoro, 5-trifluoromethyl and 5-cyano;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each hydrogen; alternatively R³ is hydrogen and R⁴ is ethyl;
or a pharmaceutically acceptable salt thereof.

5. The method of claim 1, wherein
R¹ is selected from the group consisting of hydrogen, halogen, trifluoromethyl and cyano;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

6. The method of claim 5, wherein
R¹ is selected from the group consisting of hydrogen, halogen, trifluoromethyl and cyano;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 6 membered, saturated or aromatic ring structure, optionally containing one to two additional heteroatoms independently selected from the group consisting of O, N and S;
or a pharmaceutically acceptable salt thereof.

7. The method of claim 6, wherein
R¹ is hydrogen;
X-Y is —S—CH—;
A is —CH₂—;
R² is hydrogen;
R³ and R⁴ are taken together with the nitrogen atom to which they are bound to form a 5 membered ring structure selected from the group consisting of pyrrolidinyl and imidazolyl;
or a pharmaceutically acceptable salt thereof.

8. The method of claim 2, wherein the compound of formula (I) is selected from the group consisting of
N-(benzo[b]thien-3-yl methyl)-sulfamide;
N-[(5-chlorobenzo[b]thien-3-yl)methyl]-sulfamide;
N-(3-benzofuranylmethyl)-sulfamide;
N-[(5-fluorobenzo[b]thien-3-yl)methyl]-sulfamide;
N-(1-benzo[b]thien-3-ylethyl)-sulfamide;
N-(1-naphthalenylmethyl)-sulfamide;
N-[(2-methyl-3-benzofuranyl)methyl]-sulfamide;
N-[(5-bromobenzo[b]thien-3-yl)methyl]-sulfamide;
N-[(4-bromobenzo[b]thien-3-yl)methyl]-sulfamide;
N-[(7-fluorobenzo[b]thien-3-yl)methyl]-sulfamide;
N-[(1-methyl-1H-indol-3-yl)methyl]-sulfamide;
N-[(4-trifluoromethylbenzo[b]thien-3-yl)methyl]-sulfamide;
N-[(4-cyanobenzo[b]thien-3-yl)methyl]-sulfamide;
N-[(benzo[b]thien-3-yl)methyl]-sulfamoylpyrrolidine;
N-[(benzo[b]thien-3-yl)methyl]-N′-ethylsulfamide;
imidazole-1-sulfonic acid [(benzo[b]thien-3-yl)methyl]-amide;
and pharmaceutically acceptable salts thereof.

9. The method of claim 1, wherein the compound of formula (I) is selected from the group consisting of N-(benzo[b]thien-3-ylmethyl)-sulfamide; N-[(5-fluorobenzo[b]thien-3-yl)methyl]-sulfamide; and pharmaceutically acceptable salts thereof.

10. A method for a glucose related disorder comprising administering to a subject in need thereof, a therapeutically effective amount of a compound selected from the group consisting of N-(benzo[b]thien-3-ylmethyl)-sulfamide and pharmaceutically acceptable salts thereof.

11. The method of claim 1, wherein the glucose related disorder is selected from the group consisting of elevated glucose levels and Type II diabetes mellitus.

12. The method of claim 10, wherein the glucose related disorder is selected from the group consisting of elevated glucose levels and Type II diabetes mellitus.

13. A method for a lipid related disorder comprising administering to a subject in need thereof, a therapeutically effective amount of a compound of formula (I) 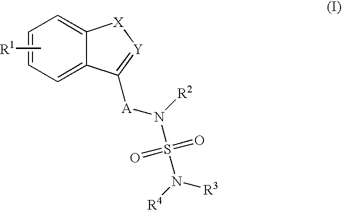
wherein
R¹ is selected from the group consisting of hydrogen, halogen, hydroxy, methoxy, trifluoromethyl, nitro and cyano;
X-Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

14. The method of claim 13, wherein
R¹ is selected from the group consisting of hydrogen, halogen, trifluoromethyl, cyano and nitro;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen, methyl and ethyl;
or a pharmaceutically acceptable salt thereof.

15. The method of claim 14, wherein
R¹ is selected from the group consisting of hydrogen, halogen, trifluoromethyl and cyano;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each independently selected from the group consisting of hydrogen and ethyl;
or a pharmaceutically acceptable salt thereof.

16. The method of claim 15, wherein
R¹ is selected from the group consisting of hydrogen, 5-chloro, 5-fluoro, 5-bromo, 4-bromo, 7-fluoro, 5-trifluoromethyl and 5-cyano;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each hydrogen; alternatively R³ is hydrogen and R⁴ is ethyl;
or a pharmaceutically acceptable salt thereof.

17. The method of claim 13, wherein
R¹ is selected from the group consisting of hydrogen, halogen, trifluoromethyl and cyano;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

18. The method of claim 17, wherein
R¹ is selected from the group consisting of hydrogen, halogen, trifluoromethyl and cyano;
X-Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 6 membered, saturated or aromatic ring structure, optionally containing one to two additional heteroatoms independently selected from the group consisting of O, N and S;
or a pharmaceutically acceptable salt thereof.

19. The method of claim 18, wherein
R¹ is hydrogen;
X-Y is —S—CH—;
A is —CH₂—;
R² is hydrogen;
R³ and R⁴ are taken together with the nitrogen atom to which they are bound to form a 5 membered ring structure selected from the group consisting of pyrrolidinyl and imidazolyl;
or a pharmaceutically acceptable salt thereof.

20. The method of claim 14, wherein the compound of formula (I) is selected from the group consisting of
N-(benzo[b]thien-3-yl methyl)-sulfamide;
N-[(5-chlorobenzo[b]thien-3-yl)methyl]-sulfamide;
N-(3-benzofuranylmethyl)-sulfamide;
N-[(5-fluorobenzo[b]thien-3-yl)methyl]-sulfamide;
N-(1-benzo[b]thien-3-ylethyl)-sulfamide;
N-(1-naphthalenylmethyl)-sulfamide;
N-[(2-methyl-3-benzofuranyl)methyl]-sulfamide;
N-[(5-bromobenzo[b]thien-3-yl)methyl]-sulfamide;
N-[(4-bromobenzo[b]thien-3-yl)methyl]-sulfamide;
N-[(7-fluorobenzo[b]thien-3-yl)methyl]-sulfamide;
N-[(1-methyl-1H-indol-3-yl)methyl]-sulfamide;
N-[(4-trifluoromethyl benzo[b]thien-3-yl)methyl]-sulfamide;
N-[(4-cyanobenzo[b]thien-3-yl)methyl]-sulfamide;
N-[(benzo[b]thien-3-yl)methyl]-sulfamoylpyrrolidine;
N-[(benzo[b]thien-3-yl)methyl]-N′-ethylsulfamide;
imidazole-1-sulfonic acid [(benzo[b]thien-3-yl)methyl]-amide;
and pharmaceutically acceptable salts thereof.

21. The method of claim 13, wherein the compound of formula (I) is selected from the group consisting of N-(benzo[b]thien-3-ylmethyl)-sulfamide; N-[(5-fluorobenzo[b]thien-3-yl)methyl]-sulfamide; and pharmaceutically acceptable salts thereof.

22. A method for a lipid related disorder comprising administering to a subject in need thereof, a therapeutically effective amount of a compound selected from the group consisting of N-(benzo[b]thien-3-ylmethyl)-sulfamide and pharmaceutically acceptable salts thereof.

23. The method of claim 13, wherein the lipid related disorder is selected from the group consisting of elevated triglyceride levels and low HDL cholesterol levels.

24. The method of claim 22, wherein the lipid related disorder is selected from the group consisting of elevated triglyceride levels and low HDL cholesterol levels.
